(19) United States
(12) Patent Application Publication
(10) Pub. No.: US 2007/0254091 A1
(43) Pub. Date: Nov. 1, 2007

(54) System and method for electrostatic-assisted spray coating of a medical device

(75) Inventors: Gerald Fredrickson, Westford, MA (US); Lan Pham, Nashua, NH (US); James Feng, Maple Grove, MN (US); Narin Anderson, Eden Prairie, MN (US); Tom Eidenschink, Rogers, MN (US); Tim O'Connor, (US)

Correspondence Address: KENYON & KENYON LLP, 1500 K STREET N.W., SUITE 700, WASHINGTON, DC, 20005, US

(73) Assignee: BOSTON SCIENTIFIC SCIMED, INC.

(21) Appl. No.: 11/412,774

(22) Filed: Apr. 28, 2006

(51) Int. Cl. A61L 33/00; B05D 1/02; B05B 5/025

(52) U.S. Cl. 427/2.24; 427/421.1; 118/620

(57) ABSTRACT

A system and method for the electrostatic spray application of a coating material onto a medical device. The coating material is electrically charged and an atomizer is used to atomize the coating material, creating electrically charged droplets which coat the medical device. In alternate embodiments, a swirl atomizer, a pressure atomizer, an ultrasound atomizer, a rotary atomizer, and an effervescent atomizer are used to atomize the coating material.

### TECHNICAL FIELD

The present invention relates to the application of coating material to medical devices.

### BACKGROUND

Coatings are often applied to implantable medical devices to increase their effectiveness or safety. These coatings may provide a number of benefits including reducing the trauma suffered during the insertion procedure, facilitating the acceptance of the medical device into the target site, or improving the effectiveness of the device.

A coating that serves as a therapeutic agent is one such way in which the coating on a medical device can improve its effectiveness. This type of coating on the medical device allows for localized delivery of therapeutic agents at the site of implantation and avoids the problems of systemic drug administration, such as producing unwanted effects on parts of the body which are not being treated, or not being able to deliver a high enough concentration of therapeutic agent to the afflicted part of the body.

Expandable stents are one specific example of medical devices that can be coated. Expandable stents are tubular structures formed in a mesh-like pattern designed to support the inner walls of a lumen, such as a blood vessel. These stents are typically positioned within a lumen and then expanded to provide internal support for the lumen. Because the stent comes into direct contact with the inner walls of the lumen, stents have been coated with various compounds and therapeutics to enhance their effectiveness. The coating on these stents may contain a drug or biologically active material which is released in a controlled fashion (including long-term or sustained release) and delivered locally to the surrounding blood vessel.

Aside from facilitating localized drug delivery, the coating on a medical device can provide other beneficial surface properties. For example, medical devices are often coated with radiopaque materials to allow for fluoroscopic visualization during placement in the body. It is also useful to coat certain devices to enhance biocompatibility or to improve surface properties such as lubricity.

For small-sized medical devices, such as a coronary artery stent, conventional spray coating methods can be inefficient. The transfer efficiency is low and much of the coating solution is lost in excessive overspraying. One way in which a coating can be applied more efficiently is to electrostatically spray the coating substance onto the device. In this method, which is also known as electrospray or electrohydrodynamic spray (and used interchangeably with electrostatic spray herein), an electrical potential difference is generated between the coating material and the target with the resulting electrostatic forces causing the coating material to atomize into fine, highly charged droplets which are then driven by the electric field lines towards the oppositely-charged target. For example, U.S. Pat. No. 6,669,980 to Hansen (filed Sep. 18, 2001), which is incorporated by reference herein, describes an electrostatic spray coating method in which a medical device is coated by electrically charged droplets that are dispensed from a nozzle. The electrostatic spray coating method described by Hansen can provide up to 60% efficiency in coating a target medical device.

However, effective electrostatic spraying usually requires a coating solution with adequate electrical conductivity. Many solvents used in the coating fluid for medical devices are organic hydrocarbon solvents such as xylene, which may not be sufficiently conductive for conventional electrostatic spray techniques. Using such low electrically conductive solutions in conventional electrostatic spray techniques can produce unsteady spray plumes with non-uniform droplet sizes, which are not suitable for the process control needed in coating medical devices.

Therefore, there is a need for an electrostatic-assisted spray coating method and apparatus for coating medical devices with coating solutions of any electrical conductivity, including those having low electrical conductivity.

### SUMMARY OF THE INVENTION

The present invention is directed to an electrostatic-assisted spray coating method and apparatus that satisfies this need. In one embodiment of the invention, a method is provided for electrostatic-assisted spray coating of a medical device in which a pressure atomizer is used to atomize the coating material.

In an alternate embodiment, a method is provided for electrostatic-assisted spray coating of a medical device in which a swirl atomizer is used to atomize the coating material.

In another alternate embodiment, a method is provided for electrostatic-assisted spray coating of a medical device in which an effervescent atomizer is used to atomize the coating material.

In yet another alternate embodiment, a method is provided for electrostatic-assisted spray coating of a medical device in which a vibrating atomizer is used to atomize the coating material.

In yet another alternate embodiment, a method is provided for electrostatic-assisted spray coating of a medical device in which a rotary atomizer is used to atomize the coating material.

In another embodiment of the present invention, a system is provided for electrostatic-assisted spray coating of a medical device in which a pressure atomizer is included in the system to atomize the coating material.

In an alternate embodiment, a system is provided for electrostatic-assisted spray coating of a medical device in which a swirl atomizer is included in the system to atomize the coating material.

In another alternate embodiment, a system is provided for electrostatic-assisted spray coating of a medical device in which an effervescent atomizer is included in the system to atomize the coating material.

In yet another alternate embodiment, a system is provided for electrostatic-assisted spray coating of a medical device in which a vibrating atomizer is included in the system to atomize the coating material.

In yet another alternate embodiment, a system is provided for electrostatic-assisted spray coating of a medical device in which a rotary atomizer is included in the system to atomize the coating material.

### BRIEF DESCRIPTION OF THE DRAWINGS

FIG. 1 is a schematic and cross-sectional view of a conventional electrospraying apparatus.

FIG. 2 is a schematic and cross-sectional view of another conventional electrospraying apparatus.

FIG. 3 is a schematic and cross-sectional view of one embodiment of the system of the present invention for electrostatic-assisted spray coating of a medical device in which the system includes a pressure atomizer.

FIG. 4 is a schematic and cross-sectional view of an alternate embodiment of the system for electrostatic-assisted spray coating of a medical device in which the system includes a swirl atomizer.

FIG. 5 is an enlarged cross-sectional view of the alternate embodiment of the electrostatic-assisted spray nozzle of FIG. 4 taken at View C.

FIG. 6 is an end view of the alternate embodiment of the electrostatic-assisted spray nozzle of FIG. 5 taken at line D-D.

FIG. 7 is a side view of a vibrating atomizer included in another alternate embodiment of the system for electrostatic-assisted spray coating of a medical device.

FIG. 8 is a side view of a rotary atomizer included in another alternate embodiment of the system for electrostatic-assisted spray coating of a medical device.

FIG. 9 is a cross-sectional view of an effervescent atomizer included in another alternate embodiment of the system for electrostatic-assisted spray coating of a medical device.

### DETAILED DESCRIPTION

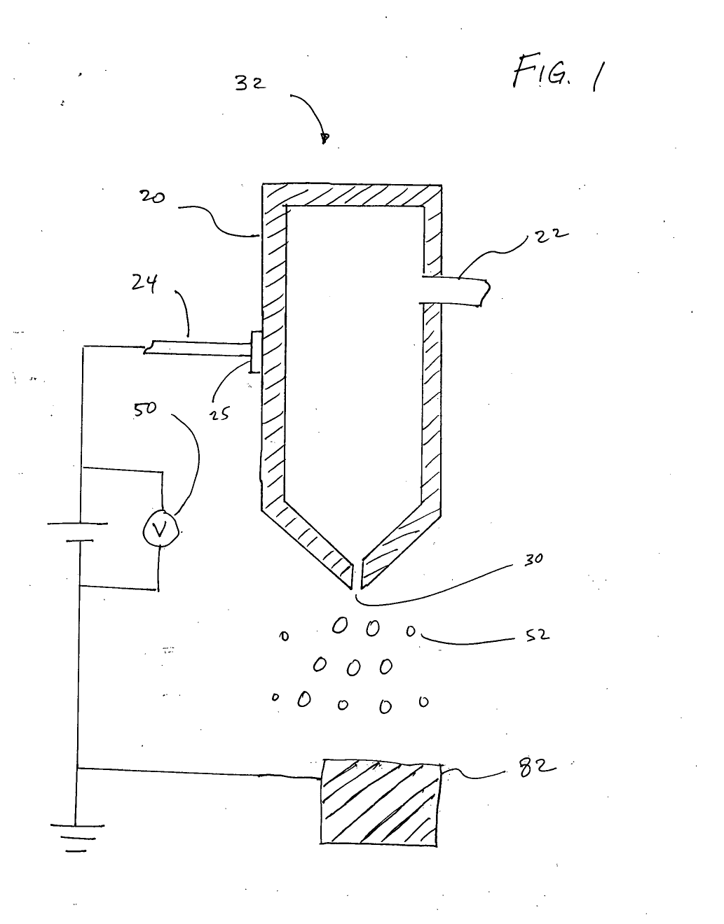

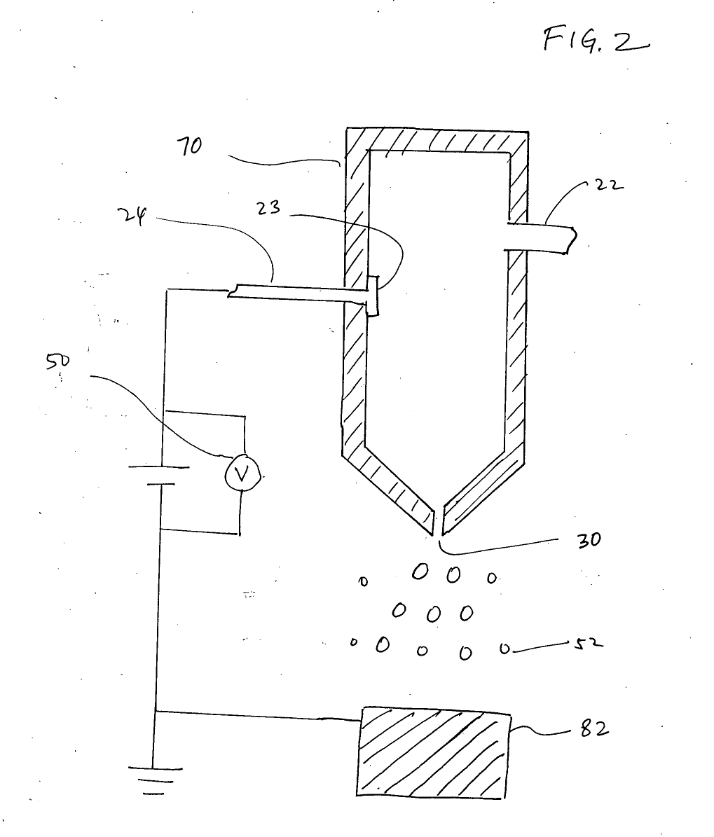

A conventional electrostatic spray apparatus is illustrated in FIG. 1. An electrostatic spray assembly 32 is shown that includes a coating material supply line 22 that supplies coating material to the spray body 20 and an electrically conducting cable 24 connected to a voltage source 50. In FIG. 1, the spray body 20 is made of an electrically conductive material. Via an electrode 25, an electric potential is conducted to the spray nozzle body 20, which then electrically charges the coating material. Alternatively, as illustrated in FIG. 2, an electrode 23 may be positioned inside an electrically insulative spray body 70. In FIG. 2, the electrode 23 receives electric current from the voltage source 50 through the cable 24, thereby injecting charge into the coating material. Additionally, one of skill in the art will appreciate that other configurations and locations for the electrode are possible, such as a ring-type electrode placed inside the nozzle near the exit orifice 30. The target 82 to be coated is held at an opposite charge (or grounded) from the coating material so that an electrical potential is created between the coating material and the target 82. The resulting electrostatic forces cause the coating material to be atomized into fine, highly charged droplets 52 which are then driven by electric field lines towards the target 82.

However, effective atomization of coating material using electrostatic forces requires the use of a coating material of sufficient electrical conductivity. Where the conductivity of the coating material is low and electrostatic atomization of the coating material is ineffective, atomization of the coating material may be enhanced by other means. For example, U.S. patent application Ser. No. 10/774,483 (filed by Worsham et al. on Feb. 10, 2004), whose entire disclosure is incorporated by reference herein, discloses an electrostatic spray coating apparatus that uses pressurized gas to enhance atomization of the charged coating fluid as the fluid emerges from the fluid nozzle orifice.

In the present invention, the system includes any type of gas-less atomizer, such as a pressure, swirl, vibrating, or rotary atomizer as described in more detail below, in which the coating material is not entrained into jets of gas. Alternatively, as also described in more detail below, the system may include an effervescent atomizer to assist in atomization of the coating material.

One of ordinary skill in the art would appreciate that enhancing atomization by using an atomizer in association with an electrostatic sprayer will allow coating material of any electrical conductivity to be used, including those having low electrical conductivity, such as a xylene solution, which has a conductivity of less than 10⁻¹⁴ S/cm, or a methyl ethyl ketone (MEK) solution, which has a conductivity of less than 10⁻⁷ S/cm.

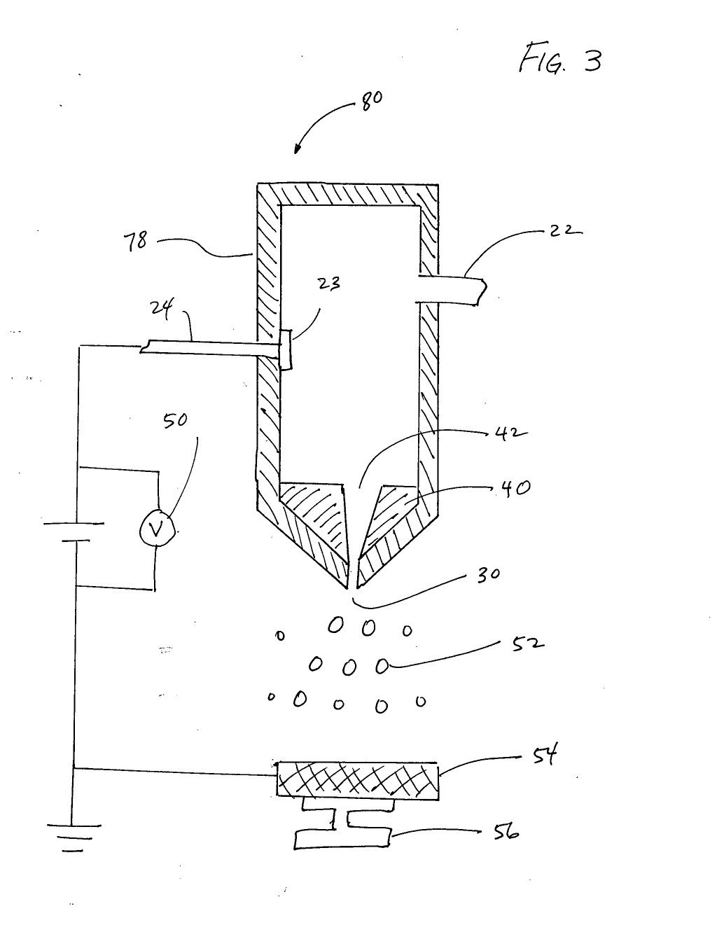

In a first embodiment of the present invention illustrated in FIG. 3, a medical device 54 to be coated with a coating material is held by a target holder 56. The medical device 54 in this instance is a coronary stent that is to be coated with a fluid containing a therapeutic agent. Non-limiting examples of other medical devices include catheters, guide wires, balloons, filters (e.g., vena cava filters), stents, stent grafts, vascular grafts, intraluminal paving systems, pacemakers, electrodes, leads, defibrillators, joint and bone implants, vascular access ports, intra-aortic balloon pumps, heart valves, sutures, artificial hearts, neurological stimulators, cochlear implants, retinal implants, and other devices that can be used in connection with therapeutic coatings. Such medical devices are implanted or otherwise used in body structures such as the coronary vasculature, esophagus, trachea, colon, biliary tract, urinary tract, prostate, brain, lung, liver, heart, skeletal muscle, kidney, bladder, intestines, stomach, pancreas, ovary, uterus, cartilage, eye, bone, and the like.

The target holder 56 may hold the medical device by any number of means, such as the stent holders described in U.S. patent application Ser. No. 10/198,094, whose entire disclosure is incorporated by reference herein. In addition to holding the medical device 54 in a position suitable for coating applications, the medical device holder 56 can also function as an electrode maintaining the medical device 54 at a first electrical potential. In certain embodiments, the medical device holder 56 functions as the electrode to maintain the medical device 54 at a first electrical potential while minimizing masking of the medical device 54 to allow for greater coating coverage. However, in another embodiment, the medical device 54 itself can be electrically connected at a first potential without using the holder 56 as an electrode.

In this first embodiment, the nozzle assembly 80 includes a coating material supply line 22 that supplies coating material to the nozzle body 78 and an electrode 23, which is connected to a voltage source 50 by an electrically conducting cable 24. A second electric potential is conducted to the electrode 23, which then electrically charges the coating material. The nozzle assembly 80 also includes a high pressure fluid atomizer 40 that is well known in the art. The pressure atomizer 40 has a fluid passageway 42 in communication with the fluid in the nozzle body 78 and a nozzle exit orifice 30 of very small diameter ranging from 0.001 inches to 0.015 inches. The ejection of fluid from the small orifice 30 under high pressure causes the fluid to atomize into small droplets 52. Because the droplets 52 are electrically charged, they repel each other and are driven by electrical field lines towards the oppositely charged medical device 54. One of skill in the art will appreciate that there are other designs for pressure atomizers which atomize fluid by ejecting the fluid through a small orifice under high pressure. For example, the pressure atomizer 40 can be used in conjunction with a plunger-type apparatus (not shown) that can increase the pressure of the coating material within the nozzle body 78.

One of ordinary skill in the art would understand that the necessary voltage potential difference between the electrode 23 and the medical device 54 will vary depending upon the size of the medical device 54, distance between the exit orifice 30 of the nozzle body 78 and the medical device 54, and electrical conductivity of the coating material. However, a potential difference between the electrode 23 and the medical device 54 in the range of 2,000 volts to 40,000 volts should be sufficient for efficient transfer of the coating material to the target medical device.

The nozzle body 78 may be made of an electrically conductive material such as stainless steel or an electrically insulative material. The electrically conducting cable 24 may be affixed to the electrode (or nozzle body) by an electrically conductive coupling, or by any other electrically conductive means that are well known to one of ordinary skill in the art, such as soldering, welding or securing with a fastener. Alternatively, if the nozzle body 78 is made of an electrically conductive material, the nozzle body 78 may serve as the electrode to electrically charge the coating material contained in the nozzle body 78, and no separate electrode 23 is necessary. An electrically conductive nozzle body 78 may be electrically connected via an electrically conducting cable to a voltage source 50.

The medical device 54 may have an electrically conductive primer coating (such as silver, salt, or conductive polymers) applied to it before undergoing electrostatic spraying to enhance its electrostatic attraction for low electrically conductive coating materials. This primer coating may be particularly useful in applying the method and apparatus of the present invention to non-metallic or non-conducting medical devices.

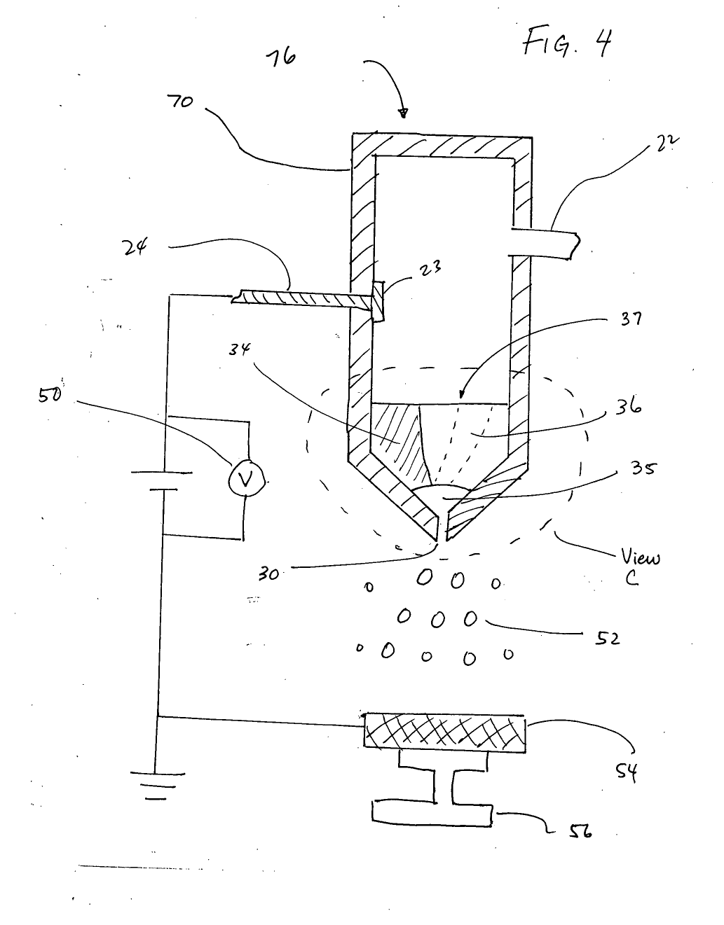

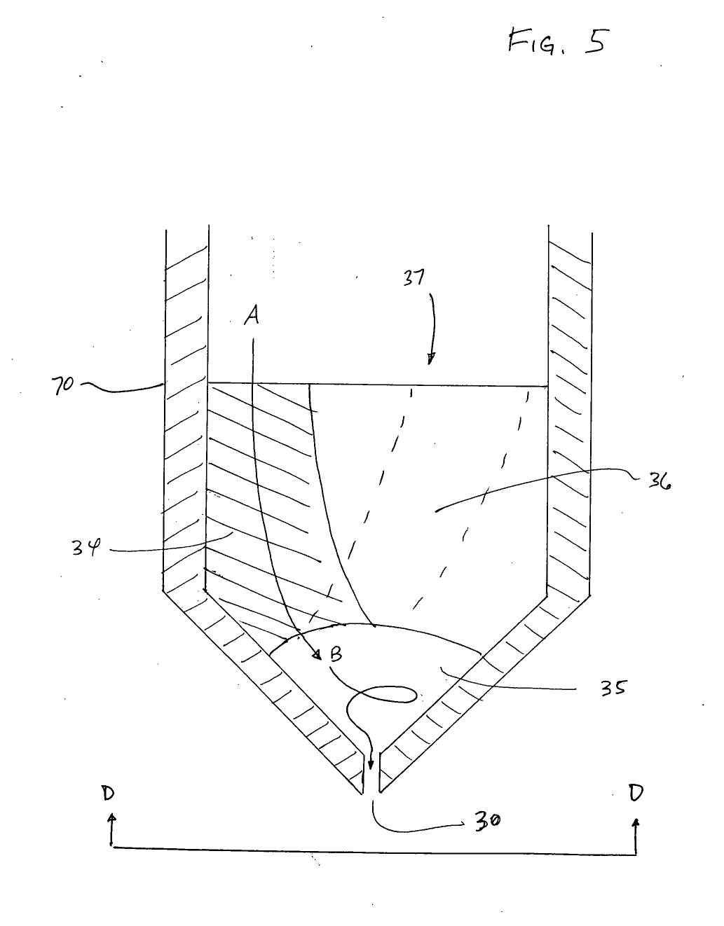

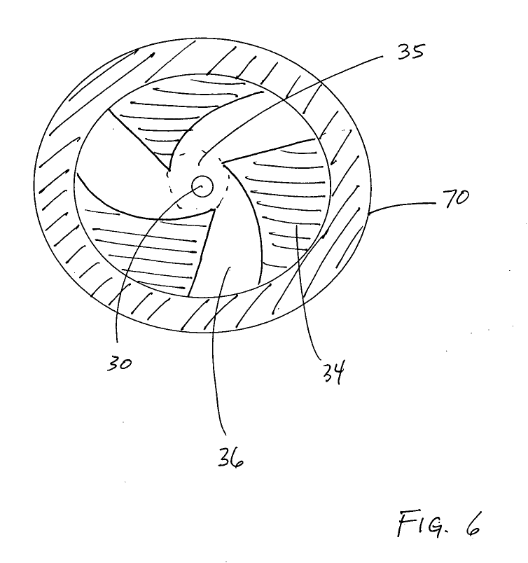

In an alternate embodiment, as illustrated in FIGS. 4-6, the nozzle assembly 76 includes a swirl atomizer 37 that is well known in the art. The swirl atomizer 37 comprises of one or more substantially tangential turbulence channels 36 formed by inner walls 34. The flow of fluid through the turbulence channels 36 has the effect of imparting rotational motion to the fluid (in the direction of arrow A in FIG. 5) as it enters the swirl chamber 35. The fluid rotates inside the swirl chamber 35 (in the direction of arrow B in FIG. 5) and emerges from the nozzle exit orifice 30. As the rotating fluid emerges from the nozzle exit orifice 30, centrifugal force causes the cone or ligaments of fluid to break up into small droplets 52. Because the coating material particles or droplets 52 are electrically charged by electrode 23, they repel each other and are driven by electrical field lines towards the oppositely charged medical device 54. One of skill in the art will appreciate that there are other designs for swirl atomizers which atomize fluid by imparting rotational motion to the fluid inside a nozzle.

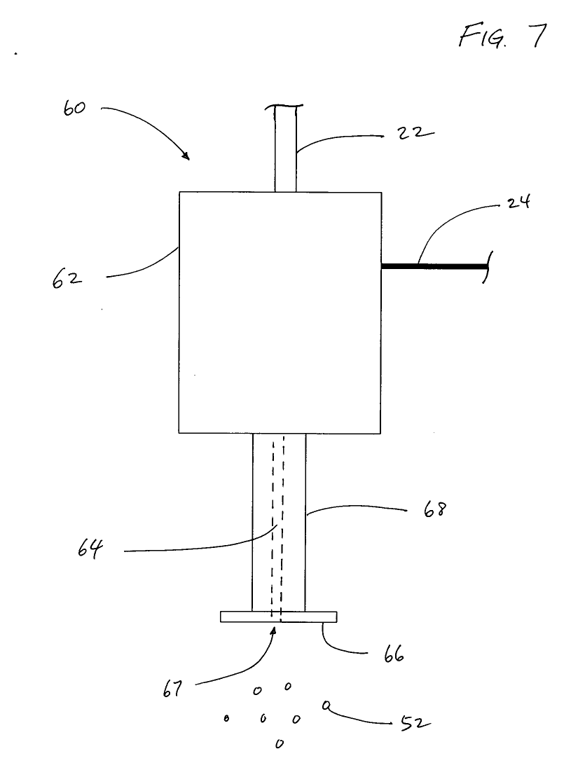

In another alternate embodiment, as illustrated in FIG. 7, the nozzle assembly 60 includes a vibrating atomizer 62 that is well known in the art. In this embodiment, a tube-shaped horn 68 on the vibrating atomizer 62 is made to vibrate at ultrasonic frequencies. The coating material is electrically charged by an electrode (not shown) within the vibrating atomizer 62 via an electrically conducting cable 24. The coating material is introduced into the vibrating atomizer 62 through coating material supply line 22 and fed through an axially extending feed channel 64 within the horn 68. The coating material then exits through exit orifice 67 and flows onto a vibrating atomizing surface 66. Vibrational energy causes the coating material to be atomized into droplets 52. Because the droplets 52 are electrically charged, they repel each other and are driven by electrical field lines towards the oppositely charged medical device (not shown). The AEROGEN™ atomizer and the atomizers described in U.S. patent application Ser. No. 11/073,198 entitled “Method of Coating a Medical Appliance Utilizing a Vibrating Mesh Nebulizer, a System for Coating a Medical Appliance, and a Medical Appliance Produced by the Method” to McMorrow; and Ser. No. 11/073,197 entitled “Method of Producing Particles Utilizing a Vibrating Mesh Nebulizer for Coating a Medical Appliance, a System for Producing Particles, and a Medical Appliance” by Behan, McMorrow, and O'Connor (which are both incorporated by reference herein and which are commonly assigned to the assignee of the instant application) are several of the many types of vibrating or ultrasonic atomizers that could be used in the present invention.

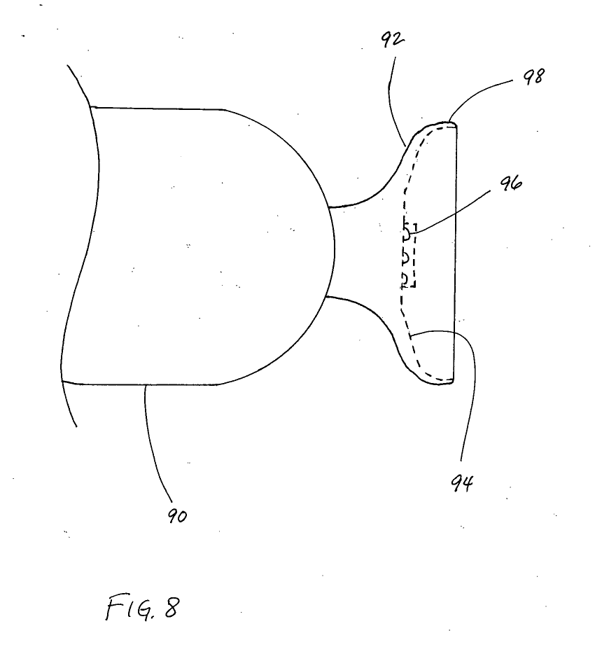

In yet another alternate embodiment, as illustrated in FIG. 8, the nozzle assembly includes a rotary atomizer 90 that is known in the art. In this embodiment, the rotary atomizer 90 has a rapidly rotating, frustro-conically shaped rotary cup 92. The coating material is electrically charged by an electrode (not shown) within the rotary atomizer 90, or by electrically charging the rotary cup 92 by connecting it to a voltage source. On the interior of the rotary cup 92 is a flow surface 94 onto which the coating material is delivered through outlet orifices 96 near the center of the rotary cup 92. Under centrifugal force, the coating material flows in an outward direction in a thin sheet along the interior flow surface 94. The peripheral edge 98 of the cup 92 is generally convexly arcuate, directing the flow of coating material in a more axial direction before being expelled from the edge of the rotary cup to form a spray plume of atomized coating material.

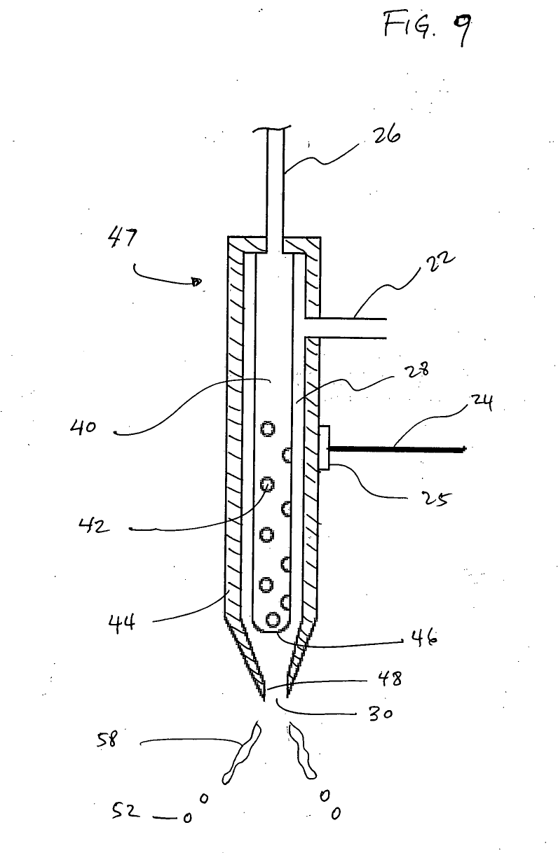

In yet another alternate embodiment, as illustrated in FIG. 9, the nozzle assembly 47 is an effervescent atomizer. In this embodiment, a stream of gas is introduced into an inner tube 40 through a gas supply line 26 which is in fluid communication with the inner tube 40. Coating material, supplied through supply line 22, is introduced into an annular space 28 defined by the inner tube 40 and the concentric outer tube 44 of the nozzle body. Towards the downstream tip 46 of the inner tube 40, there are openings 42 which allow the gas to exit the inner tube 40 and enter the coating material, thus forming gas bubbles in the coating material. The coating material and the gas bubbles exit through a nozzle orifice 30. As the gas bubbles exit the nozzle orifice 30, the bubbles force the coating material against the inside wall 48 of the orifice 30. The layer of coating material on the orifice wall 48 is ejected from the orifice 30 in thin sheets or ligaments 58 of coating material which disintegrate into small droplets 52. The gas bubbles are also thought to rapidly increase in volume as they emerge from the orifice 30, providing additional force that shatters the coating material into small droplets 52. One of skill in the art will appreciate that there are other designs for effervescent atomizers which atomize fluid by introducing gas bubbles into the fluid as it exits the nozzle orifice. One of skill in the art will also appreciate that a variety of gases, including nitrogen or air, could be used to introduce bubbles in the fluid.

The therapeutic agent may be any pharmaceutically acceptable agent such as a non-genetic therapeutic agent, a biomolecule, a small molecule, or cells.

Exemplary non-genetic therapeutic agents include anti-thrombogenic agents such heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, zotarolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis(2-aminoethyl)ethyleneglycol-N,N,N′,N′-tetraacetic acid and mixtures thereof; antibiotics such as gentainycin, rifampin, iminocyclin, and ciprofolxacin; antibodies including chimeric antibodies and antibody fragments; anesthetic agents such as lidocaine, bupivacaine, and ropivacaine; nitric oxide; nitric oxide (NO) donors such as linsidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; inhibitors of heat shock proteins such as geldanamycin; angiotensin converting enzyme (ACE) inhibitors; beta-blockers; bAR kinase (bARKct) inhibitors; phospholamban inhibitors; protein-bound particle drugs such as ABRAXANE™; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include serca-2 protein, monocyte chemoattractant proteins (“MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin⁻) cells including Lin⁻CD34⁻, Lin⁻CD34⁺, Lin⁻cKit⁺, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells, adult cardiac fibroblasts+5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells.

Any of the therapeutic agents may be combined to the extent such combination is biologically compatible.

Any of the above mentioned therapeutic agents may be incorporated into a polymeric coating on the medical device or applied onto a polymeric coating on a medical device. The polymers of the polymeric coatings may be biodegradable or non-biodegradable. Non-limiting examples of suitable non-biodegradable polymers include polystrene; polyisobutylene copolymers, styrene-isobutylene block copolymers such as styrene-isobutylene-styrene tri-block copolymers (SIBS) and other block copolymers such as styrene-ethylene/butylene-styrene (SEBS); polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; cellulosic polymers such as cellulose acetate; polymer dispersions such as polyurethane dispersions (BAYHDROL®); squalene emulsions; and mixtures and copolymers of any of the foregoing.

Non-limiting examples of suitable biodegradable polymers include polycarboxylic acid, polyanhydrides including maleic anhydride polymers; polyorthoesters; poly-amino acids; polyethylene oxide; polyphosphazenes; polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc-calcium phosphate.

Such coatings used with the present invention may be formed by any method known to one in the art. For example, an initial polymer/solvent mixture can be formed and then the therapeutic agent added to the polymer/solvent mixture. Alternatively, the polymer, solvent, and therapeutic agent can be added simultaneously to form the mixture. The polymer/solvent/therapeutic agent mixture may be a dispersion, suspension or a solution. The therapeutic agent may also be mixed with the polymer in the absence of a solvent. The therapeutic agent may be dissolved in the polymer/solvent mixture or in the polymer to be in a true solution with the mixture or polymer, dispersed into fine or micronized particles in the mixture or polymer, suspended in the mixture or polymer based on its solubility profile, or combined with micelle-forming compounds such as surfactants or adsorbed onto small carrier particles to create a suspension in the mixture or polymer. The coating may comprise multiple polymers and/or multiple therapeutic agents.

The coating is typically from about 1 to about 50 microns thick. In the case of balloon catheters, the thickness is preferably from about 1 to about 10 microns, and more preferably from about 2 to about 5 microns. Very thin polymer coatings, such as about 0.2-0.3 microns and much thicker coatings, such as more than 10 microns, are also possible. It is also within the scope of the present invention to apply multiple layers of polymer coatings onto the medical device. Such multiple layers may contain the same or different therapeutic agents and/or the same or different polymers. Methods of choosing the type, thickness and other properties of the polymer and/or therapeutic agent to create different release kinetics are well known to one in the art.

The medical device may also contain a radio-opacifying agent within its structure to facilitate viewing the medical device during insertion and at any point while the device is implanted. Non-limiting examples of radio-opacifying agents are bismuth subcarbonate, bismuth oxychloride, bismuth trioxide, barium sulfate, tungsten, and mixtures thereof.

While the present invention has been described with reference to what are presently considered to be preferred embodiments thereof, it is to be understood that the present invention is not limited to the disclosed embodiments or constructions. On the contrary, the present invention is intended to cover various modifications and equivalent arrangements. For example, the coating material may comprise a flowable solid material, such as a powder, in lieu of a fluid, as long as the flowable solid coating material can be reliably fed through the dispensing device and accept a charge imparted by the second potential. The present invention is also suitable for use in a high speed automated medical device coating apparatus. In as much as this invention references dispensed particles, these particles can be in the form of droplets with or without entrained solids at various levels of evaporation. Furthermore, these particles can be dispensed as a solution, a suspension, an emulsion, or any type flowable material as described above.

While the various elements of the disclosed invention are described and/or shown in various combinations and configurations, which are exemplary, other combinations and configurations, including more, less or only a single embodiment, are also within the spirit and scope of the present invention.

What is claimed is:

1. A method for electrostatic-assisted spray coating of a medical device, comprising the steps of:
(a) providing a medical device;
(b) providing a coating discharge nozzle, wherein the nozzle includes an electrode and an orifice;
(c) introducing a coating material into the coating discharge nozzle;
(d) applying an electrical potential difference between the medical device and the electrode to electrically charge the coating material;
(e) atomizing the electrically charged coating material into electrically charged coating material particles with a gas-less atomizer; and
(f) discharging the electrically charged particles of coating material from the orifice of the discharge nozzle onto the medical device.

2. The method of claim 1, wherein the gas-less atomizer is a swirl atomizer.

3. The method of claim 1, wherein the gas-less atomizer is a pressure atomizer.

4. The method of claim 1, wherein the gas-less atomizer is a vibrating atomizer.

5. The method of claim 1, wherein the gas-less atomizer is a rotary atomizer.

6. The method of claim 1, wherein the medical device is a stent.

7. The method of claim 1, wherein the step of applying an electrical potential difference between the medical device and the electrode includes electrically connecting the electrode to a voltage source at a first electrical potential and electrically connecting the medical device at a second electrical potential.

8. The method of claim 1, wherein the coating material is of low electrical conductivity.

9. The method of claim 1, wherein the coating material contains a therapeutic agent.

10. The method of claim 1, further comprising the step of applying an electrically conductive primer coating to the medical device.

11. A method for electrostatic-assisted spray coating of a medical device, comprising the steps of:
(a) providing a medical device;
(b) providing a coating discharge nozzle, wherein the nozzle includes an electrode and an orifice;
(c) introducing a coating material into the coating discharge nozzle;
(d) applying an electrical potential difference between the medical device and the electrode to electrically charge the coating material;
(e) atomizing the electrically charged coating material into electrically charged coating material particles with an effervescent atomizer; and
(f) discharging the electrically charged particles of coating material from the orifice of the discharge nozzle onto the medical device.

12. The method of claim 11, wherein the medical device is a stent.

13. The method of claim 11, wherein the coating material is of low electrical conductivity.

14. The method of claim 11, wherein the coating material contains a therapeutic agent.

15. The method of claim 11, further comprising the step of applying an electrically conductive primer coating to the medical device.

16. A system for electrostatic-assisted spray coating of a medical device, comprising:
(a) a medical device;
(b) a coating discharge nozzle adapted to receive coating material, wherein the nozzle includes an electrode and a nozzle orifice;
(c) a means for applying an electrical potential difference between the medical device and the electrode to electrically charge the coating material; and
(d) a gas-less atomizer for atomizing the electrically charged coating material into electrically charged coating material particles.

17. The system of claim 16, wherein the gas-less atomizer is a pressure atomizer.

18. The system of claim 16, wherein the gas-less atomizer is a swirl atomizer.

19. The system of claim 16, wherein the gas-less atomizer is a vibrating atomizer.

20. The system of claim 16, wherein the gas-less atomizer is a rotary atomizer.

21. The system of claim 16, wherein the medical device is a stent.

22. The system of claim 16, wherein the coating material contains a therapeutic agent.

23. The system of claim 16, wherein the medical device is coated with an electrically conductive primer coating.

24. A system for electrostatic-assisted coating of a medical device, comprising:
(a) a medical device;
(b) a coating discharge nozzle adapted to receive coating material, wherein the nozzle includes an electrode and a nozzle orifice;
(c) a means for applying an electrical potential difference between the medical device and the electrode to electrically charge the coating material; and
(d) an effervescent atomizer for atomizing the electrically charged coating material into electrically charged coating material particles.

25. The system of claim 24, wherein the medical device is a stent.

26. The system of claim 24, wherein the coating material contains a therapeutic agent.

27. The system of claim 24, wherein the medical device is coated with an electrically conductive primer coating.
